(19) United States
(12) Patent Application Publication
(10) Pub. No.: US 2024/0147581 A1
(43) Pub. Date: May 2, 2024

(54) Thawing Device For Frozen Biological Material

(71) Applicant: Thermo Fisher Scientific (Asheville) LLC, Asheville, NC (US)

(72) Inventors: Nathan Norman Lang, Newington, NH (US); Warren C. Prouty, Newington, NH (US)

(21) Appl. No.: 18/498,866

(22) Filed: Oct. 31, 2023

Related U.S. Application Data
(60) Provisional application No. 63/381,600, filed on Oct. 31, 2022.

(51) Int. Cl. H05B 3/22

(52) U.S. Cl. CPC H05B 3/22

(57) ABSTRACT

A thawing device, comprising: a housing; a first heater plate assembly supported by the housing; at least one first heater thermally coupled to the first heater plate assembly; a second heater plate assembly supported by the housing and opposing the first heater plate assembly to define a bag receiving region between opposing faces of the first heater plate assembly and the second heater plate assembly; at least one second heater thermally coupled to the second heater plate assembly; the thawing device being arranged such that at least one of the first heater plate assembly and the second heater plate assembly is moveable relative to the other of the first heater plate assembly and the second heater plate assembly such that the first heater plate assembly and the second heater plate assembly engage with, and apply heat to, respective opposite sides of the at least one bag containing frozen liquid.

### RELATED APPLICATIONS

The present application claims priority to and the benefit of U.S. patent application No. 63/381,600, “Thawing Device For Frozen Biological Material” (filed Oct. 31, 2022). All foregoing applications are incorporated herein by reference in their entireties for any and all purposes.

### FIELD OF THE INVENTION

This invention relates generally to thawing devices and, more particularly, to a laboratory thawing device and related methods for thawing frozen biological material.

### BACKGROUND OF THE INVENTION

Thawing devices are commonly used in laboratory and other settings to thaw frozen biological material, such as plasma, blood, platelets, tissue, cells, molecular reagents and antibodies, by way of example, which has been stored for a period of time in a frozen state under cryogenic temperatures. The biological material is typically stored in a sample container or vessel, such as a sample bag, often referred to as a “cryobag.” Cryobags can vary in physical size and volume depending on the particular application for which the biological material is to be used. For example, common cryobag volumes include 250 ml, 500 ml and 750 ml bags, although cryobags of other volumes are possible as well.

In use, the cryobag containing the frozen biological material may be placed in a thawing device which heats the bag sufficiently to thaw the biological material within the bag. In that regard, it is important to control the thawing process, including the thawing rate and the temperature profile across the cryobag, to avoid damaging the thawed biological material contained within the bag.

To thaw the biological material contained in the cryobag, the sample bag is typically first disposed in an overwrap or barrier bag which may take the form of a reversibly sealable pouch. The barrier bag is used to capture any condensation produced or biological material that may leak from the cryobag during the thawing operation to prevent contamination of the thawing device.

Prior to the thawing process, the cryobag, and its barrier bag if used, are placed in direct or indirect contact with a heat source of the thawing device. The heat source may be stationary to maintain engagement with the sample bag. However, placing the sample bag in contact with a stationary heat source does not provide the controlled environment for optimal regulation of the thawing process, especially as the biological material changes phase from solid to liquid. Furthermore, a stationary heat source can cause an uneven temperature profile across the sample bag, resulting in certain parts of the sample bag and the biological material becoming hotter than others as the biological material changes phase from solid to liquid. Over time, this can cause hot spots across the sample bag that can damage (i.e., denature) the biological material. To this end, the longer it takes to thaw the biological material, the greater the chance that damaging hot spots develop across the sample bag and the biological material stored therein.

In another known thawing approach, a thawing device is provided with a pair of opposing heater plates which define a receiving space for a cryobag containing frozen biological material disposed between opposing faces of the pair of heater plates. In this known thawing approach, one of the heater plates is typically fixed, and the other heater plate is movable toward the fixed heater plate. In this thawing approach, the heater plates engage with, and thaw, the cryobag which is disposed in the receiving space between the pair of plates. Different methods or mechanisms for agitating the cryobag during the thawing process may be provided.

However, these known thawing devices having one of the heater plates being movable toward and away from each other, heater plate which may be fixed, and also providing agitation of the cryobag during the thawing process, tend to be relatively complicated in structure and operation.

Therefore, a need exists to provide a thawing device for thawing frozen biological material contained in a cryobag efficiently without damaging the biological material contained within the bag such as by subjecting the biological material to excessive heat, for example. Furthermore, it is desirable that the thawing device agitate the biological material during the thawing process, especially as the biological material changes phase from solid to liquid, to improve the thermal exchange in the sample bag and thus the thawing rate without the damaging effects of hot spots.

### SUMMARY OF THE INVENTION

The present invention overcomes the foregoing and other shortcomings and drawbacks of known thawing devices for thawing frozen biological material stored in a sample bag. While the present invention will be discussed in connection with certain embodiments, it will be understood that the present invention is not limited to the specific embodiments described herein.

In one embodiment of the present invention, a thawing device is provided that is configured to thaw frozen liquid contained in at least one sample bag. The thawing device includes a housing, a first heater plate assembly supported by the housing and at least one first heater that is thermally coupled to the first heater plate assembly. The thawing device also includes a second heater plate assembly supported by the housing that opposes the first heater plate assembly to define a bag receiving region between opposing faces of the first heater plate assembly and the second heater plate assembly, with at least one second heater thermally coupled to the second heater plate assembly.

At least one scissors mechanism is operatively coupled to the first heater plate assembly and the second heater plate assembly, and an actuator is operatively coupled to the at least one scissors mechanism. The actuator, in a first mode of operation, is operable to move the first heater plate assembly and a second heater plate assembly toward each other to engage with, and apply heat to, respective opposite sides of the at least one bag containing frozen liquid, with the frozen liquid being thawed to form thawed liquid within the at least one bag which the first and second heaters are operated.

In a second mode of operation, the actuator is operable to move the first heater plate assembly and the second heater plate assembly away from each other to disengage from the opposite sides of the at least one bag containing the thawed liquid.

In one embodiment, the first heater plate assembly includes a first heater carrier plate and a first thermally conductive heater plate resiliently biased relative to the first heater carrier plate, and the second heater plate assembly includes a second heater carrier plate and a second thermally conductive heater plate resiliently biased relative to the second heater carrier plate.

According to one embodiment, the first thermally conductive heater plate is resiliently biased away from the first heater carrier plate, and the second thermally conductive heater plate is resiliently biased away from the second heater carrier plate.

According to one aspect of the present invention, at least one first temperature sensor is thermally coupled to the first thermally conductive heater plate, and at least one second temperature sensor is thermally coupled to the second thermally conductive heater plate.

In one embodiment, at least one sample temperature sensor is supported by one of the first and second thermally conductive heater plates, and a pressure sensor is supported by one of the first or second thermally conductive heater plates.

In one embodiment, a plurality of first heaters are thermally coupled to the first thermally conductive heater plate to define a plurality of different first heater zones associated with the first thermally conductive heater plate, and a plurality of second heaters are thermally coupled to the second thermally conductive heater plate to define a plurality of different second heater zones associated with a second thermally conductive heater plate.

In one embodiment, a plurality of first sample temperature sensors are supported by the first thermally conductive heater plate, with each of the plurality of first sample temperature sensors being associated with a respective one of a plurality of different first heater zones. A plurality of second sample temperature sensors are supported by the second thermally conductive heater plate, with each of the plurality of second sample temperature sensors being associated with a respective one of the plurality of different second heater zones.

Various additional features and advantages of the invention will become more apparent to those of ordinary skill in the art upon review of the following detailed description of one or more illustrative embodiments taken in conjunction with the accompanying drawings.

### BRIEF DESCRIPTION OF THE DRAWINGS

The accompanying drawings, which are incorporated in and constitute a part of this specification, illustrate one or more embodiments of the invention and, together with the general description given above and the detailed description given below, serve to describe the one or more embodiments of the invention.

FIG. 1A is a perspective view of a thawing device in accordance with an exemplary embodiment of the present invention, showing an outer housing and a Human Machine Interface (HMI) of the thawing device.

FIG. 1B is a view similar to FIG. 1A, showing a view with the outer housing and HMI of the thawing device removed.

FIG. 2 is a side partial cross-sectional view of the thawing device of FIG. 1A in an “OPEN” mode, illustrating details of a scissors mechanism provided on one side of the thawing device and an actuator for operating the scissors mechanism.

FIG. 3 is a view similar to FIG. 2, illustrating details of a scissors mechanism provided on an opposite side of the thawing device and the actuator of FIG. 2 tor operating the scissors mechanism.

FIG. 4 is a view similar to FIG. 3, illustrating a bag containing frozen liquid being suspended in a bag receiving region between opposing faces of front and rear heater plate assemblies.

FIG. 4A is an enlarged view of the encircled area 4A in FIG. 4.

FIG. 5 is a side partial cross-sectional view of the thawing device of FIG. 1A in a “HEAT” mode, illustrating opposing faces of the front and rear heater plate assemblies engaging with, and applying heat to, respective opposite sides of the bag containing frozen liquid to form thawed liquid within the bag.

FIG. 5A is an enlarged view of the encircled area 5A in FIG. 5.

FIG. 6 is a partial rear perspective view of the thawing device shown in FIG. 1A.

FIG. 7 is a schematic representation of an exemplary control system implemented in the thawing device of FIG. 1A.

FIG. 8 is a diagrammatic view of a thermally conductive heater plate according to one embodiment of the present invention, showing multiple heater zones of the thermally conductive heater plate.

### DETAILED DESCRIPTION OF THE INVENTION

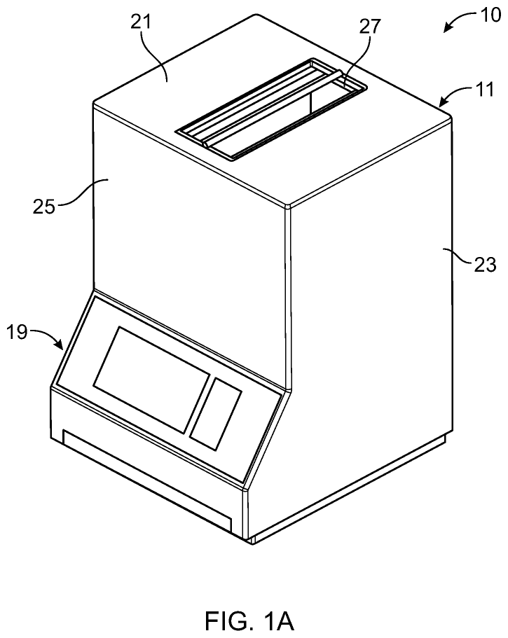

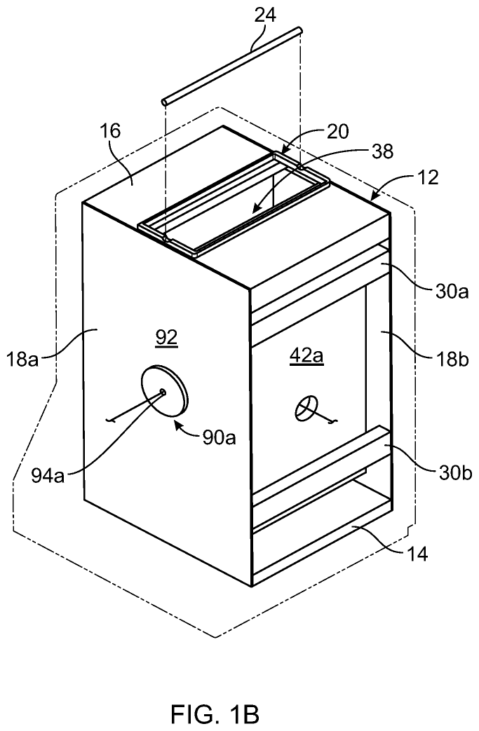

Referring now to the figures, and to FIGS. 1A and 1B in particular, an exemplary thawing device 10, otherwise often referred to as a “cryo thawer”, is shown in accordance with an embodiment of the present invention.

As shown in FIGS. 1-5, the thawing device 10 includes an outer housing or shell 11 (FIG. 1A) and an inner housing 12 (FIG. 1B). As shown in FIG. 1B, the inner housing 12 has a base or bottom wall 14, an opposing top wall 16, a pair of opposing side walls 18a-b, a front wall (not shown), and a rear wall (not shown). The various walls 14, 16, 18a-b, and the front and rear walls (not shown), may be joined together to form an enclosure for the internal mechanics of the thawing device 10 as will be described in greater detail below.

As shown in FIG. 1A, the outer housing 11 includes a base or bottom wall (not shown), an opposing top wall 21, a pair of opposing side walls 23 (one shown) located adjacent the front and rear walls (not shown) of the inner housing 12, a front wall 25 located adjacent the side wall 18a of the inner housing 12 and a rear wall (not shown) located adjacent the side wall 18b of the inner housing 12. The outer housing 11 supports a Human Machine Interface (HMI) 19 which is configured to receive user inputs and to display various operating parameters of the thawing device 10 during user of the thawing device 10 in a thawing operation as will be described in greater detail below.

The HMI 19 may include video or alphanumeric displays, a touch screen, a speaker, and any other suitable audio and visual indicators capable of providing data to the user. The HMI 19 may also include input devices and controls such as an alphanumeric keyboard, a pointing device, keypads, pushbuttons, control knobs, a USB port, microphones, or any other suitable input device or control capable of accepting commands or input from the user and transmitting the entered input to the controller of the thawing device 10 as described below.

As used herein, the terms “bottom,” “top,” “side,” “front,” and “rear” are used herein for description purposes only in view of the figures, and these terms are not intended to be limiting in any way. For example, while the thawing device 10 may be supported on a laboratory benchtop or table during use with the front wall 25 of the outer housing 11 facing the user, it is equally possible that the thawing device 10 may be oriented with the rear wall of the outer housing 11 (not shown) facing the user such that the rear wall now becomes the front wall and the front wall now becomes the rear wall. It is equally possible that the thawing device 10 may be arranged sideways, i.e. rotated 90°, with either of the side walls 23 of the outer housing 11 now facing the user such that one of the side walls 23 now becomes the front wall and the other of the side walls 23 now becomes the rear wall. Moreover, while the thawing device 10 is shown in the figures in a vertical orientation with the top wall 21 of the outer housing 11 being located above the bottom wall (not shown), the thawing device 10 may, alternatively, be positioned in a horizontal orientation to rest on one of its sides, with one of the side walls 23 now becoming the bottom wall, the other of the side walls 23 becoming the top wall. In yet another possible orientation of the thawing device 10, the thawing device 10 may again be positioned in a horizontal orientation to rest on the rear wall (not shown) such that the rear wall (not shown) now becomes the bottom wall, and the front wall 25 now becomes the top wall.

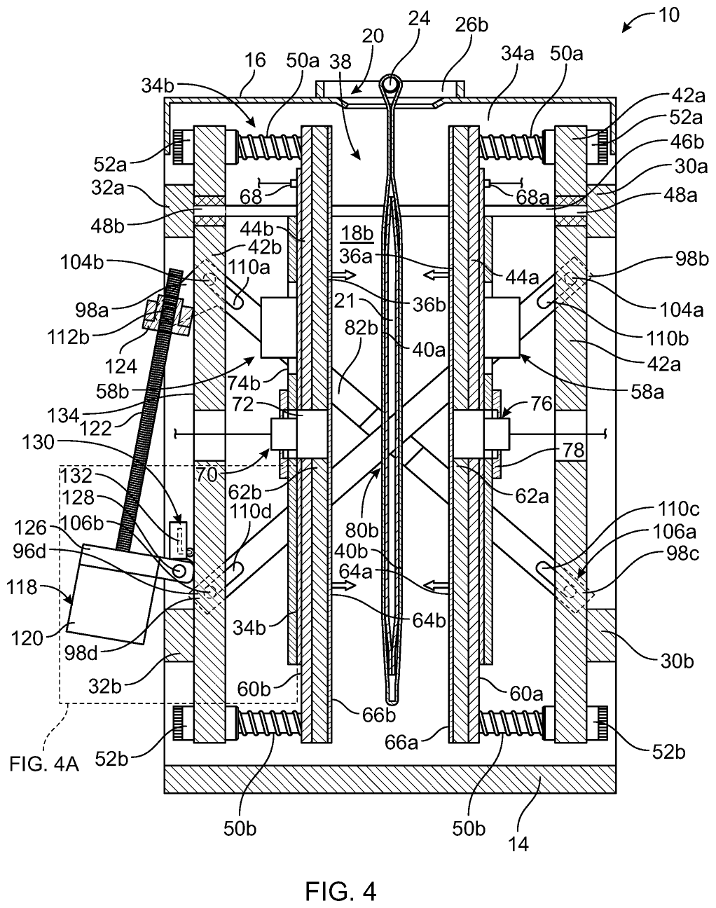

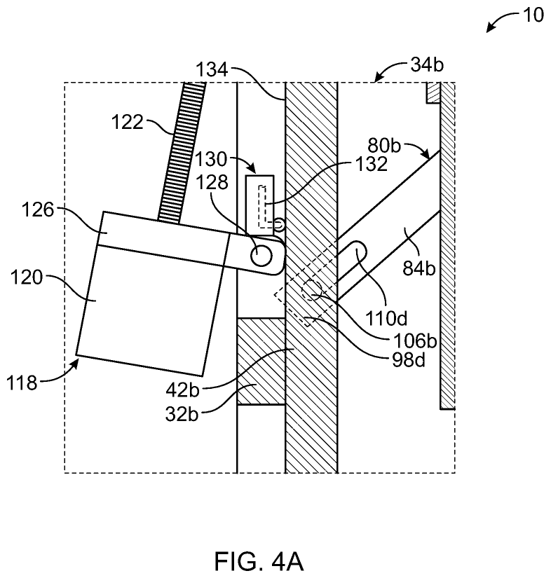

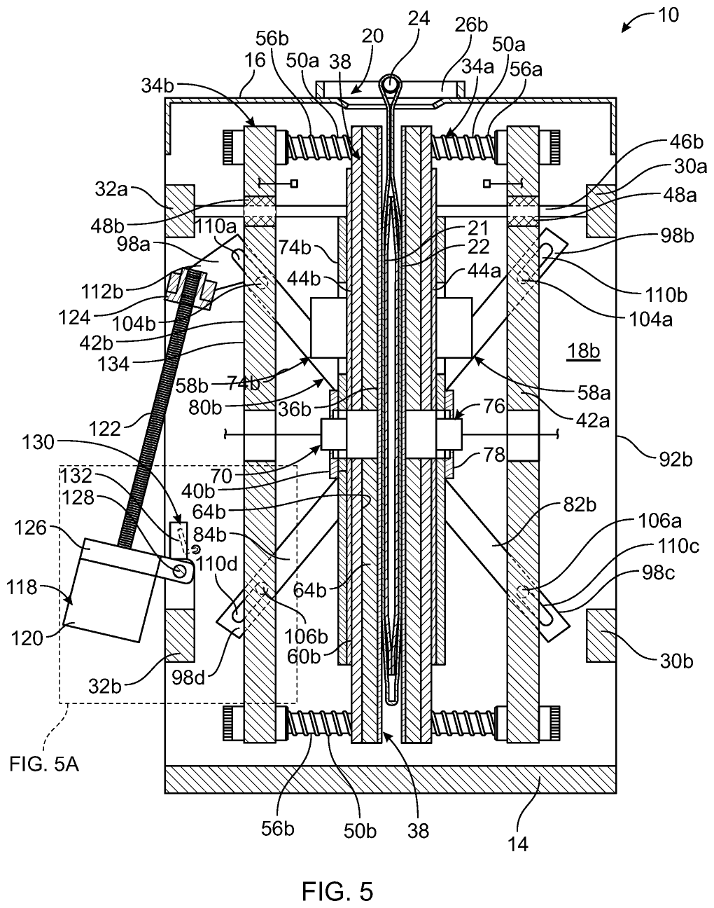

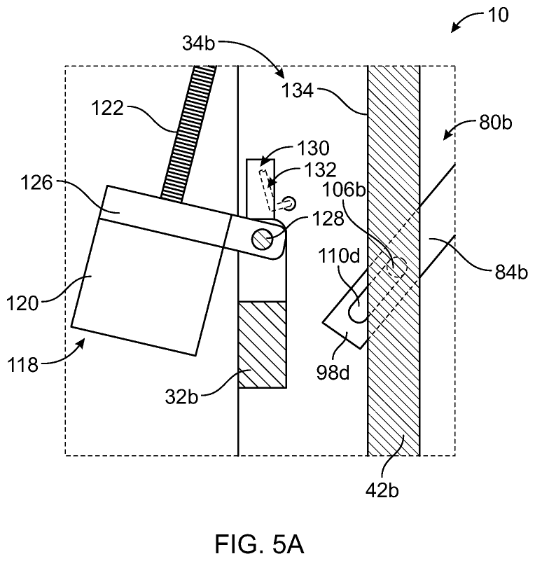

As shown in FIGS. 1-6, the top wall 21 of the outer housing 11 and the top wall 16 of the inner housing 12 are provided with respective elongated openings or slots 27 and 20 which are configured so that a user can insert a sample vessel containing a frozen biological material 21, such as a cryobag 22 (FIGS. 4-5) containing a frozen biological material, referred herein as “sample,” into the thawing device 10 through the slots 27 and 20 by suspending the cryobag 22 by a horizontally oriented hanging rod 24 which is removably located in a hanging rod support frame 29 provided adjacent the slots 27 and 20 as will be described in greater detail below.

Prior to inserting the cryobag 22 into the thawing device 10, the cryobag 22 may be inserted into, and sealed within, a barrier bag 28 as shown in FIGS. 4 and 5 which is designed to capture any condensation or biological material that may leak from the cryobag 22 during the thawing operation to prevent contamination of the thawing device 10.

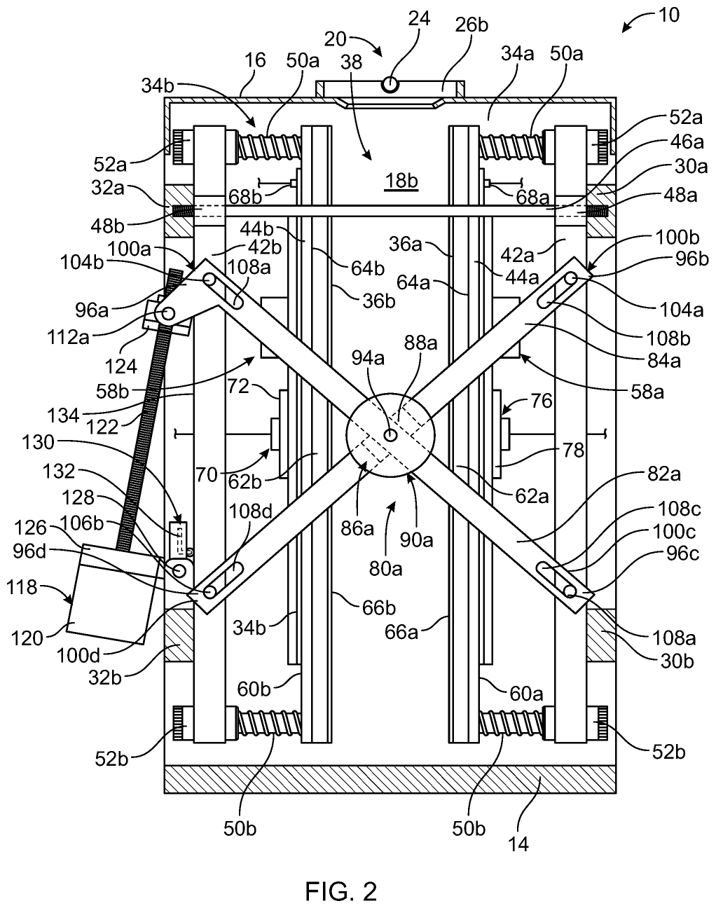

Referring now to FIGS. 2-5, the exemplary thawing device 10 includes upper and lower front, horizontally oriented, cross-braces, respectively, 30a-b, which extend between, and are fastened at their respective opposite ends to the side walls 18a-b adjacent the front wall 25 of the inner housing 11. Similarly, the thawing device 10 includes upper and lower rear, horizontally oriented, cross-braces, respectively, 32a-b, which extend between, and are fastened at their respective opposite ends to the side walls 18a-b of the inner housing 11 adjacent the rear wall (not shown).

In the exemplary embodiment of the thawing device 10 as shown, the thawing device 10 is provided with a pair of opposing and vertically oriented heater plate assemblies, respectively, 34a-b, which will be referred to herein as a front heater plate assembly 34a and a rear heater plate assembly 34b. Again, as set forth above, the terms “front” and “rear” are used herein for description purposes only in view of the figures, and these terms are not intended to be limiting in any way.

Referring still to FIGS. 2-5, the front and rear heater plate assemblies 34a-b are positioned inwardly of the respective cross-braces 30a-b, 32a-b, and have respective opposing and vertically oriented faces 36a-b that define a receiving space 38 for at least one sample vessel containing frozen biological material, such as at least one cryobag 22 as shown in FIGS. 4 and 5, that is inserted into the receiving space 38.

As will be described in greater detail below, the opposing faces 36a-b of the respective front and rear heater plate assemblies 34a-b are operated to move horizontally into engagement with respective opposite sides 40a-40b of the cryobag 22 or barrier bag 28 during a thawing operation so as to apply heat to the opposite sides 40a-b of the cryobag 22 or barrier bag 28 to thaw the frozen biological material contained within the cryobag 22.

According to one aspect of the present invention as shown in FIGS. 2-5, each of the front and rear heater plate assemblies 34a-b is mounted in a vertical orientation within the thawing device 10 so as to be movable horizontally toward each other to engage with, and apply heat to, the respective opposite sides 40a-b of the cryobag 22 or barrier bag 28 during a thawing operation. When the thawing operation is complete, the front and rear heater plate assemblies 34a-b are also mounted so as to be movable horizontally away from each other to disengage from the opposite sides 40a-b of the cryobag 22 or barrier bag 28 which now contains thawed biological liquid within the bag 22. As will be described in greater detail below, the front and rear heater plate assemblies 34a-b may also move back and forth repeatedly during certain phases of the thawing process to agitate the sample during thawing.

Once the front and rear heater plate assemblies 34a-b are disengaged from the opposite sides 40a-b of the bag 22 or overwrap bag 28, the bag 22 now containing the thawed biological liquid can be removed from the thawing device 10 by lifting the hanging rod 24 and the bag 22 upwardly, and overwrap bag 28 if used, until the bag 22 fully exits the slot 20. The bag 22 of thawed biological liquid may then be removed from the overwrap bag 28 if one is used.

In one embodiment of the present invention as shown in FIGS. 2-5, the front heater plate assembly 34a includes a vertically oriented front heater carrier plate 42a mounted inwardly of the pair of front cross-braces 30a-b, and a vertically oriented front thermally conductive heater plate 44a that is mounted parallel to and resiliently biased relative to the front heater carrier plate 42a as will be described in greater detail below.

Similarly, the rear heater plate assembly 34b includes a vertically oriented rear heater carrier plate 42b mounted inwardly of the pair of rear cross-braces 32a-b, and a vertically oriented rear thermally conductive heater plate 44b that is mounted parallel to and resiliently biased relative to the rear heater carrier plate 42a as will be described in greater detail below.

As shown in FIGS. 2-6, the thawing device 10 includes a pair of spaced apart, horizontally oriented, elongated guide shafts 46a-b that are threadably fastened at their respective opposite ends to the front and rear upper cross-braces 30a, 32a located in an upper portion of the housing 12. Each of the front and rear heater carrier plates 42a-b includes a respective pair of spaced apart linear ball bearings 48a-b mounted to a respective upper portion of each of the front and rear heater plate carriers 42a-b. The pair of elongated guide shafts 46a-b extend through the respective linear ball bearings 48a-b so that each of the front and rear heater carrier plates 42a-b is mounted for horizontal sliding movement toward and away from each other along the elongated guide shafts 46a-b. The guide shafts 46a-b provide proper alignment and linear motion of the front and rear heater carrier plates 42a-b horizontally along the guide shafts 46a-b as the front and rear heater carrier plates 42a-b move toward and away from each other during operation of the thawing device 10 as will be described in greater detail below.

In one embodiment, each of the front and rear thermally conductive heater plates 44a-b includes a pair of spaced apart, horizontally oriented, upper guide shafts 50a and a pair of spaced apart, horizontally oriented, lower guide shafts 50b threadably fastened to each of the respective front and rear thermally conductive heater plates 44a-b. In one embodiment, each of the upper and lower guide shafts 48a-b, 50a-b is internally threaded at one respective inner end to receive a respective fastener (not shown) extending from the front and rear thermally conductive heater plates 44a-b.

As shown in FIGS. 2-5, each of the front and rear heater carrier plates 42a-b is provided with a pair of spaced apart upper linear ball bearings 52a mounted to a respective upper portion of front and rear heater carrier plates 42a-b, and a pair of spaced apart lower linear ball bearings 52b mounted to a respective lower portion of the front and rear heater carrier plates 42a-b. The upper and lower guide shafts 50a-b are mounted to extend through the respective upper and lower linear ball bearings 52a-b so that the front and rear thermally conductive heater plates 44a-b are mounted for horizontal sliding movement toward and away the respective front and rear heater carrier plates 42a-b during operation of the thawing device 10 as described in detail below.

In one embodiment, the front thermally conductive heater plate 44a is biased horizontally away from the front heater carrier plate 42a in an inward direction toward the rear thermally conductive heater plate 44b by resilient members, such as compression springs 56a, supported by each of the upper and lower guide shafts 50a-b. The compression springs 56a are positioned between the front heater carrier plate 42 and the front thermally conductive heater plate 44a so that, in response to an outwardly directed pressure applied to the face 36a of the front thermally conductive heater plate 44a, the front thermally conductive heater plate 44a is compressed, against the biasing force of the compression springs 56a, in a direction toward the front heater carrier plate 42a as shown in FIG. 5. When that outwardly directed pressure is removed from the face 36a of the front thermally conductive heater plate 44a, the compression springs 56a again bias the front thermally conductive heater plate 44a in an inward direction away from the front heater carrier plate 42a as shown in FIGS. 2-4.

Similarly, the rear thermally conductive heater plate 44b is biased horizontally away from the rear heater carrier plate 42b in an inward direction toward the front thermally conductive heater plate 44a by resilient members, such as compression springs 56b, supported by each of the upper and lower guide shafts 50a-b. The compression springs 56b are positioned between the rear heater carrier plate 42b and the rear thermally conductive heater plate 44b so that, in response to an outwardly directed pressure applied to the face 36b of the rear thermally conductive heater plate 44b, the rear thermally conductive heater plate 44b is compressed, against the biasing force of the compression springs 56b, in a direction toward the rear heater carrier plate 42b as shown in FIG. 5. When that outwardly directed pressure is removed from the face 36b of the rear thermally conductive heater plate 44b, the compression springs 56b again bias the rear thermally conductive heater plate 44b in an inward direction away from the rear heater carrier plate 42b as shown in FIGS. 2-4.

In one embodiment, again referring to FIGS. 2-5, the front and rear thermally conductive heater plates 44a-b each comprises a rectangular metal plate, such as made of aluminum, having a height dimension of 12 in., a width dimension of 6 in. and a thickness dimension of ⅛″ to ¼″, although other dimensions and materials for the front and rear thermally conductive plates 44a-b are possible as well depending on the particular thawing operation requirements.

Heat is applied to each of the front and rear thermally conductive heater plates 44a-b by respective front and rear heaters 58a-b which are thermally coupled to each of the respective front and rear thermally conductive heater plates 44a-b. In an exemplary embodiment, each of the front and rear heaters 58a-b may be a 300W heater which may be wired with dual circuits for 115V and 230V use. Each front and rear heater 58a-b is thermally coupled by acrylic pressure sensitive adhesive (PSA) to respective outer faces 60a-b of each of the front and rear thermally conductive heater plates 44a-b, with each outer face 60a and 60b being a face of the front and rear thermally conductive heater plates 44a-b that confronts the respective front and rear walls (not shown) of the housing 12. In one embodiment, each of the front and rear heaters 58a-b may be a silicone mat resistance heater that is commercially available from Watlow, located in Austin, TX, as Model No. 040089M00S, although other heater types are possible as well as will be understood by those of ordinary skill in the art.

As shown in FIGS. 2-5, each of the front and rear thermally conductive heater plates 44a-b has a respective rectangular, thermally conductive, foam pad 62a-b mounted to respective inner faces 64a-b of the front and rear thermally conductive plates 44a-b. In one embodiment, each of the thermally conductive foam pads comprises silicone rubber sheet or thermally conductive foam that is commercially available from Bellofram, located in Newell, WV, as Model No. 8304, although other thermally conductive pad materials are possible as well as will be understood by those of ordinary skill in the art.

Each of the thermally conductive foam pads 62a-b is overwrapped with a thermoplastic protective film 66a-b (FIGS. 2-4), which, in one embodiment, is a polytetrafluoroethylene (“ptfe”) film. The thermoplastic protective film 66a-b is secured to each of the respective front and rear thermally conductive heater plates 44a-b by brackets (not shown) which are secured to the respective outer faces 60a-b of the front and rear thermally conductive heater plates 44a-b.

Further referring to FIGS. 2-5, each of the front and rear thermally conductive plates 44a-b includes a respective front and rear heater plate temperature sensor 68a-b that is thermally coupled to the respective front and rear thermally conductive heater plates 44a-b. The front and rear heater plate temperature sensors 68a-b are provided to monitor the respective temperatures of the front and rear thermally conductive heater plates 44a-b during a thawing operation. In one embodiment, the front and rear heater plate temperature sensors 68a-b may comprise resistance temperature detectors (“RTD's”) that are mounted to the respective outer faces 60a-b of the front and rear thermally conductive heater plates 44a-b by adhering the RTD's to the front and rear thermally conductive heater plates 44a-b using PSA, or alternatively, are mechanically attached via pockets in the front and rear thermally conductive heater plates 44a-b and held in place (sandwiched) with the respective foam pads 62a-b. While RTD's are described in one embodiment, other temperature sensors are possible as well as will be understood by those of ordinary skill in the art.

As will be described in detail below, the temperature of the cryobag 22 is monitored during the thawing operation to control operation of the thawing device 10 while the cryobag 22 is being thawed. In one embodiment as shown in FIGS. 2-5, at least one of the front and rear thermally conductive heater plates 44a-b supports a sample temperate sensor 70 (one shown). In the exemplary embodiment shown in FIGS. 2-5, the sample temperature sensor 70 is shown supported by the rear thermally conductive heater plate 44b and may comprise a digital temperature sensor that is commercially available from Texas Instruments, located in Dallas TX, as Model No. TMP117, although other types of sample temperature sensors are possible as well as will be appreciated by those of ordinary skill in the art. A thermistor spring cap 72 is provided to support the sample temperature sensor 70, with the thermistor spring cap 72 being mounted to the outer face 60b of the rear thermally conductive plate 44b by a rear backing plate 74b as shown in the exemplary embodiment.

As will also be described in greater detail below, the pressure being applied to an inner face 64a-b of at least one of the front and rear thermally conductive heater plates 44a-b is monitored during a thawing operation. In the embodiment shown in the figures, the front thermally conductive heater plate 44a supports a pressure sensor 76 which is configured to monitor a pressure applied to the inner face 64a of the front thermally conductor heater plate 44a, either by engagement of the front thermally conductive foam pad 62a with one side 40a of the cryobag 22 as shown in FIG. 5, or by engagement of the front thermally conductive foam pad 62a with the rear thermally conductive foam pad 62b (the engagement not shown in the figures) as will be described in greater detail below.

The pressure sensor 76 may comprise a piezo resistive force sensor that is commercially available from Honeywell, located in Charlotte, NC, as Part No. FSG015WNPB, although other force sensors are possible as well as will be appreciated by those of ordinary skill in the art. A pressure sensor spring cap 78 is provided to support the pressure sensor 76, with the pressure sensor spring cap 78 being mounted to the outer face 60a of the front thermally conductive plate 44a by a front backing plate 74a as shown in the exemplary embodiment.

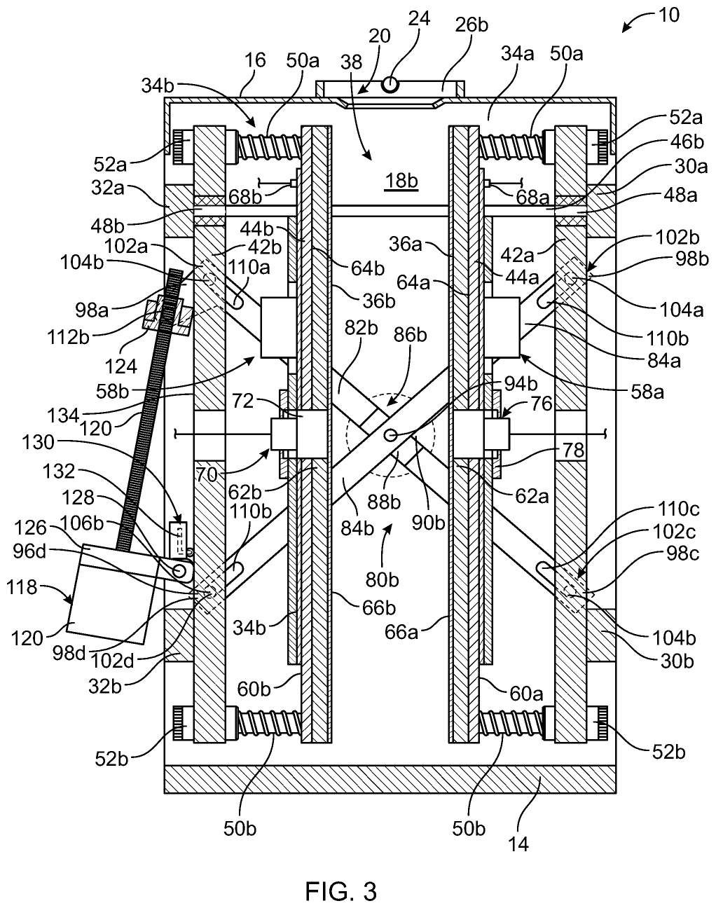

Still referring to FIGS. 2-5, movement of the front and rear heater plate assemblies 34a-b toward and away from each other is accomplished, in part, by at least one, and preferably two scissors mechanisms 80a-b which are operatively coupled to opposite sides of the front and rear heater plate assemblies 34a-b. In the exemplary embodiment shown in the figures, each of the pair of scissors mechanisms 80a-b includes respectively, two arms 82a, 84a, 82b, 84b that are generally centrally overlapped at respective pivot locations 86a-b (FIGS. 2-3) so as to be arranged in an x-configuration. In this embodiment, the cross-sectional thickness of each of the respective pair of arms 82a, 84a, 82b, 84b of a respective scissors mechanism 80a-b is reduced by a respective notch 88a-b (FIGS. 2-3) generally in the center of each arm 84b, 82a at the respective pivot location 86a-b so that the cross-sectional thickness of a respective scissors mechanism 80a-b at the overlap of the respective two arms 82a, 84a, 82b, 84b is generally equal to the cross-sectional thickness of either arm 82a, 84a, 82b, 84b. In this way, the pair of arms 82a, 84a, 82b, 84b of a respective scissor mechanism 80a-b lies in a common vertical plane, with each respective vertical plane being located on opposite sides of the front and rear heater plate assemblies 34a-b.

As shown in the exemplary embodiment of FIGS. 1-3, the respective pivot location 86a-b for each scissors mechanism 80a-b is provided by a respective linkage pivot 90a-b (FIGS. 1-3) which is mounted to a respective outer face 92a-b of each of the pair of opposite side walls 18a-b. In the exemplary embodiment, each of the linkage pivots 90a-b (FIGS. 1 and 3) includes a respective horizontally oriented dowel pin 94a-b which extends inwardly from the respective side walls 18a-b and extends through each of the respective scissors mechanisms 80a-b at the respective pivot locations 86a-b. In this way, when each of the pair of scissors mechanisms 80a-b is operated as described in greater detail below, each of the pair of arms 82a, 84a, 82b, 84b of a respective scissors mechanism 80a-b is operable to rotate in clockwise and counter-clockwise directions about the respective dowel pins 94a-b at the respective pivot locations 86a-b.

In one embodiment as shown in the figures, each of the respective pair of arms 82a, 84a, 82b, 84b has respective outer terminal ends 96a-d and 98a-d that are operatively coupled to respective opposite sides of the front and rear heater carrier plates 42a-b at four distinct locations on each of the opposite sides of the respective front and rear heater carrier plates 42a-b.

As shown in the exemplary embodiment, each respective opposite side of the front and rear heater carrier plates 42a-b includes a respective upper, outwardly extending and horizontally oriented dowel pin 104a-b, and a respective lower, outwardly extending and horizontally oriented dowel pin 106a-b. In the embodiment shown in FIGS. 2-5, respective terminal ends 96a-d, 98a-b of the pair of arms 82a, 84a, 82b, 84b of a respective scissors mechanism 80a-b includes respective elongated slots 108a-d, 110a-d which slidably engage the respective upper and lower dowel pins 104a-b, 106a-b of the pair of front and rear thermally conductive heater plates 44a-b.

During operation of the thawing device 10, as described in greater detail below, to move the front and rear heater plate assemblies 42a-b toward and away from each other, the dowel pins 104a-b, 106a-b are free to slidably travel within the respective elongated slots 108a-d, 110a-d in directions toward and away from respective inner and outer opposite ends of the respective elongated slots 108a-d, 110a-d. In particular, as the front and rear heater plate assemblies 34a-b are moved or positioned towards each other as shown in FIG. 5 in response to operation of the pair of scissors mechanisms 80a-b, the respective upper and lower dowel pins 104a-b, 106a-b of the front and rear heater plate assemblies 34a-b move simultaneously in directions toward the respective inner ends of the respective elongated slots 108a-d, 110a-d. Conversely, when the front and rear heater plate assemblies 34a-b are moved or positioned away each other as shown in FIG. 2-4 in response to operation of the pair of scissors mechanisms 80a-b, the respective upper and lower dowel pins 104a-b, 106a-b of the front and rear heater plate assemblies 34a-b move simultaneously in directions toward the respective outer ends of the respective elongated slots 108a-d, 110a-d.

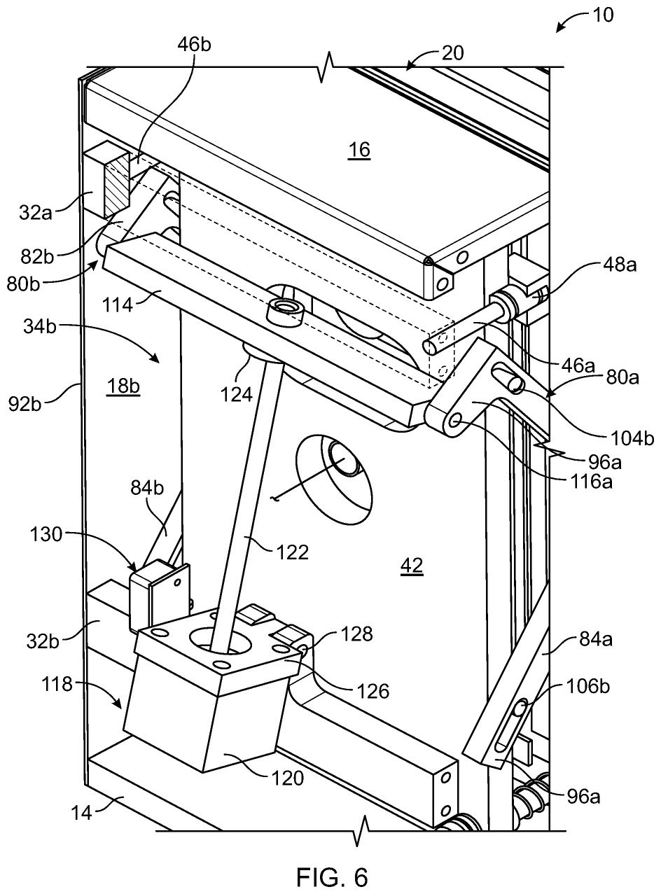

In one embodiment, each of the respective outer terminal ends 96a, 98a of the respective arms 82a-b includes a respective flange 112a-b that is rotatably coupled to opposite ends of a horizontally oriented rotatable rocker plate 114 (FIG. 6) as described in greater detail below. As shown in FIG. 6, the rocker plate 114 includes a pair of outwardly extending and horizontally oriented dowel pins 116 (one shown) which pivotally connect to a respective one of the pair of flanges 112a-b.

Further referring to FIGS. 2-6, movement of the front and rear heater plate assemblies 34a-b toward and away from each other is also accomplished, in part, by an actuator 118 that is operatively coupled to the pair of scissors mechanism 80a-b. In the exemplary embodiment, the actuator 118, in combination with the pair of scissors mechanisms 80a-b, operate together to move the front and rear heater plate assemblies 34a-b toward and away from each other during a thawing operation of the thawing device 10, as well as during agitation of the sample to achieve a more uniform thawing of the sample without hot spots as described in detail below.

In one embodiment as shown in the figures, that actuator 118 is a stepper motor 120 having an elongated ball screw 122 that is rotatably coupled to a ball nut 124 supported by the rocker plate 114. In an exemplary embodiment, the actuator 118 is supported by a stepper motor mounting plate 126 that is pivotally coupled about a horizontal axis 128 (FIG. 6) to the lower rear cross-brace 32b. In one embodiment, the stepper motor 120 may be commercially available from Nanotec, located in Auburn, MA, as Model No. LSA421S14-A-UKDE-152, although other types of actuators are possible as well as will be appreciated by those of ordinary skill in the art.

As will be described in greater detail below, when the actuator 118 is operated to move the front and rear heating plate assemblies 34a-b away from each other to an “open” position as shown in FIGS. 2-4, the stepper motor 120 is actuated so that the ball screw 122 is rotated in a clockwise direction in the ball nut 124 supported by the rocker plate 114. As the ball screw 122 is rotated in the clockwise direction, the ball nut 124 travels downwardly along the ball screw 122 in a direction toward the stepper motor 120. As the ball nut 124 travels downwardly along the ball screw 122, the rocker plate 114 also travels downwardly with the ball nut 124 in the direction of the stepper motor 120.

As this occurs, the rocker plate 114 slightly rotates in a counter-clockwise direction and pulls the respective flanges 112a-b of the pair of scissor mechanisms 80a-b downwardly with the rocker plate 114. This downward movement of the respective flanges 112a-b causes simultaneous rotation of the respective pair of arms 82a, 84a, 82b, 84b of each of the pair of scissors mechanisms 80a-b about their respective pivot locations 86a-b so that the respective upper and lower dowel pins 104a-b, 106a-b mounted to the front and rear heater carrier plates 42a-b simultaneously move in directions toward the respective outer ends of the respective elongated slots 108a-d, 110a-d. As this simultaneous movement of the respective upper and lower dowel pins 104a-d, 106a-d occurs in the respective slots 108a-d, 110a-d, the front and rear heater plate assemblies 34a-b simultaneously move outwardly to the “open” position wherein the bag receiving space 38 is opened as shown in FIGS. 2-4. As the ball nut 124 travels downwardly along the ball screw 122, the stepper motor mounting plate 126 and the stepper motor 120 supported by it, rotate slightly in a clockwise direction about the horizontal axis 128 (FIG. 6).

In one embodiment as shown in FIGS. 2-4 and 4A, a two-state limit switch 130 is supported by the lower rear cross-brace 32b. The limit switch 130 may comprise a roller lever miniature snap acting switch that is commercially available from Omron, located in Hoffman Estates, IL, as Model No. V-15G5-1C26-K, although other types of limit switches are possible as well as will be appreciated by those of ordinary skill in the art.

As shown in FIGS. 2-4 and 4A, simultaneous outward movement of the pair of front and rear heater plate assemblies 34a-b is stopped when a rocker lever 132 of the limit switch 130 engages an outer face 134 of the rear heater carrier plate 42b and is rotated in a counter-clockwise to a final position as shown in FIGS. 2-4, 4A and 6. In this final position of the rocker lever 132 of the limit switch 130, the limit switch 130 transitions from an electrically “closed” state to an electrically “open” state which terminates operation of the actuator 118 to move the front and rear heater plate assemblies 34a-b simultaneously away from each other.

As will also be described in greater detail below, when the actuator 118 is operated to move the front and rear heating plate assemblies 34a-b simultaneously toward each other to a “closed” position as shown in FIG. 5, the stepper motor 120 is actuated so that the ball screw 122 is rotated in a counter-clockwise direction in the ball nut 124 supported by the rocker plate 114. As the ball screw 122 is rotated in the counter-clockwise direction, the ball nut 124 travels upwardly along the ball screw 122 in a direction away the stepper motor 120. As the ball nut 124 travels upwardly along the ball screw 122, the rocker plate 114 also travels upwardly with the ball nut 124 in a direction away from the stepper motor 120.

As this occurs, the rocker plate 114 slightly rotates in a clockwise direction and pushes the respective flanges 112a-b of the pair of scissor mechanisms 80a-b upwardly with the rocker plate 114. This upward movement of the respective flanges 112a-b causes simultaneous rotation of the respective pair of arms 82a-b, 84a-b of each of the pair of scissors mechanisms 80a-b about their respective pivot locations 86a-b so that the respective upper and lower dowel pins 104a-b, 106a-b mounted to the front and rear heater carrier plates 42a-b simultaneously move in directions away from the respective outer ends of the respective elongated slots 108a-d, 110a-d and toward the respective inner ends of the respective elongated slots 108a-d, 110a-d. As this simultaneous movement of the respective upper and lower dowel pins 104a-b, 106a-b occurs in the respective slots 108a-d, 110a-d, the front and rear heater plate assemblies 34a-b simultaneously move inwardly to toward each other to the “closed” position shown in FIG. 5. In this “closed” position, the front and rear heater plate assemblies 34a-b engage with, and are operable to apply heat to, respective opposite sides 40a-b of the cryobag 22 positioned the bag receiving space 38 as shown in FIG. 5 during a thawing operation. As the ball nut 124 travels upwardly along the ball screw 122, the stepper motor mounting plate 126 and the stepper motor 120 supported by it, rotate slightly in a clockwise direction about the horizontal rotational axis 128.

The thawing device 10 is designed to operate in various modes of operation before, during and after a thawing operation. These modes of operation include the “CLOSED” mode, the “PREHEAT” mode, the “OPEN” mode, the “HEAT” mode, and the “AGITATION” mode. Each of these modes of operation is described in detail below.

### “CLOSED” Mode

In this normally “off” state of the thawing device 10, the front and rear heater plate assemblies 34a-b are positioned into engagement with each other. The final positions of the front and rear heater plate assemblies 34a-b in the “CLOSED” mode is established by moving the front and rear heater plate assemblies 34a-b in opposite horizontal directions toward each other without a cryobag 22 being inserted or located between the front and rear thermally conductive heater plates 44a-b. Movement of the front and rear heater plate assemblies 34a-b toward each other terminates when the pressure sensor 76 supported by the front thermally conductive heater plate 44a senses a predetermined engagement pressure or force being applied to the front thermally conductive heater plate 44a, such as a sensed pressure or force of about 0.5 kg, although other predetermined engagement pressure or force amounts are possible as well to terminate movement of the front and rear heater plate assemblies 34a-b toward each other to establish the “CLOSED” mode. Alternatively, a predetermined step count limit may be established and calibrated, and the stepper motor 120 may be operated to move the front and rear heater plate assemblies 34a-b toward each other until the predetermined step count limit is reached.

### “PREHEAT” Mode

In the “PREHEAT” mode, the front and rear heater plate assemblies 34a-b engage each other without a cryobag 22 being inserted or located between the front and rear thermally conductive heater plates 44a-b as a result of the prior “CLOSED” mode. In the “PREHEAT” mode, the front and rear heaters 58a-b are operated to heat the respective front and rear thermally conductive heater plates 44a-b to a predetermined preheat temperature, such as about 50° C., although other predetermined preheat temperatures are possible as well. As described above, and as will be described in greater detail below, the respective front and rear heater plate temperature sensors 68a-b continuously or intermittently monitor the respective temperatures of the front and rear thermally conductive heater plates 44a-b during the “PREHEAT” mode of operation.

### “OPEN” Command

In response to the thawing device receiving an “OPEN” command from a user to open the front and rear heater plates assemblies 34a-b so as to create the bag receiving space 38 between the front and rear thermally conductive heater plates 44a-b, the stepper motor 120 operates to move the front and rear heater plates assemblies 34a-b in opposite horizontal directions until the outer face 60b of the rear heater carrier plate 44b engages the limit switch 130 and moves the limit switch 130 to the “open” position as shown in FIGS. 2-4, and as described in detail above. When the limit switch 130 transitions to the “open” position, continued outward movement of the front and rear heater plate assemblies 34a-b by the stepper motor 120 is terminated. In the final positions of the front and rear heater plate assemblies 34a-b in response to the “OPEN” command, the bag receiving space 38 is established between the front and rear thermally conductive heater plates 44a-b to now receive a cryobag 22 to be thawed as shown in FIG. 4.

### “HEAT” Mode

Prior to the “HEAT” mode, the cryobag 22 is inserted through the elongated opening or slot 20 provided on the top wall 16 of the thawing device 10 and suspended in place within the bag receiving space 38 by the hanging rod 24. During the “HEAT” mode to thaw the cryobag 22 during a thawing operation, the stepper motor 120 is operated to move the front and rear heater plate assemblies 34a-b in opposite horizontal directions so that the respective front and rear thermally conductive heater plates 44a-b engage with the opposite sides 40a-b of the cryobag 22. Movement of the front and rear heater plate assemblies 34a-b toward each other terminates when the pressure sensor 76 supported by the front thermally conductive heater plate 44a senses a predetermined engagement pressure or force being applied to the front thermally conductive heater plate 44a, such as a sensed pressure or force of about 0.5 kg, although other predetermined engagement pressure or force amounts are possible as well to terminate movement of the front and rear heater plate assemblies 34a-b toward each other.

As the front and rear thermally conductive heater plates 44a-b move into engagement with the opposite sides 40a-b of the cryobag 22, the front and rear thermally conductive heater plates 44a-b may be slightly compressed toward the respective front and rear heater plate carriers 42a-b against the bias of the compression springs 56a-b, to better adapt to the outer contours of the cryobag 22, especially if the opposite sides 40a-b of the cryobag 22 are not generally parallel or there is some bulging at the opposite sides 40a-b of the cryobag 22. During the “HEAT” mode, the front and rear heaters 58a-b are operated to maintain the temperature of each of the front and rear heater plates 44a-b at a predetermined thawing temperature, such as about 50° C., although other predetermined thawing temperatures are possible as well. As described above, and as will be described in greater detail below, the respective front and rear heater plate temperature sensors 68a-b continuously or intermittently monitor the respective temperatures of the front and rear thermally conductive heater plates 44a-b during the “HEAT” mode of operation. Also during the “HEAT” mode, as will be described in greater detail below, the sample temperature sensor 70 supported by the rear thermally conductive heater plate 44b is operated to sense the temperature of the cryobag 22 during the thawing operation to control operation of the thawing device 10 while the cryobag 22 is being thawed.

### “AGITATION” Mode

During the thawing operation, the frozen biological material contained within the cryobag 22 experiences a phase change transition from a frozen solid state to a liquid state. During this phase change transition, the frozen biological material may transform into a somewhat frozen state, such as a frozen slush state. As this occurs, it is important to maintain and control a generally uniform temperature profile across the opposite sides 40a-b of the cryobag 22 to avoid damaging the thawed biological material contained within the cryobag 22 during the thawing operation.

In one embodiment of the present invention, the thawing device 10 is provided with an “AGITATION” mode to agitate the thawing biological material contained within the cryobag 22, especially when it is in the frozen slush state, to provide better control of the heat profile across the cryobag 22 during the thawing operation.

In the exemplary embodiment, the sample temperature sensor 70 is operated to sense the temperature of the sample contained within the cryobag 22 during the thawing operation. In one embodiment, when the sensed sample temperature reaches a predetermined thawing temperature, such as about 4°, for example, which is indicative that the sample within the cryobag 22 is thawing, the thawing device 10 enters the “AGITATION” mode automatically. Alternatively, the thawing device 10 may enter the “AGITATION” mode by monitoring the sample temperature sensor 70 for a “flat” line of temperature which is indicative that the sample within the cryobag 22 is thawing. In yet another alternative embodiment, the pressure sensor 76 may be monitored, and when a predetermined pressure or force drop is sensed by the pressure sensor 76, indicative that the sample within the cryobag 22 is thawing, the thawing device 10 enters the “AGITATION” mode automatically.

In the “AGITATION” mode, the stepper motor 120 is operated to move the front and rear heater plates assemblies 34a-b in a pulsed manner toward and away from each other to thereby agitate the sample contained with the cryobag 22 and to improve thermal contact of the front and rear thermally conductive heater plates 44a-b with the respective opposite sides 40a-b of the cryobag 22. The amount of movement of the front and rear heater plate assemblies 44a-b toward and away from each other during the “AGITATION” mode may be controlled in response to operation of the pressure sensor 76 as described in detail above or by using a preset step count for the stepper motor 120 in each direction during the “AGITATION” mode, for example.

In one embodiment, the sensed sample temperatures at which the “AGITATION” mode is initiated and/or the thawing operation is terminated may be selected operating parameters that are entered into the HMI 19 by the user.

When a predetermined sample temperature is sensed by the sample temperature sensor 70, which is indicative that the sample is sufficiently thawed within the cryobag 22, the “AGITATION” mode is terminated and the front and rear heater plate assemblies 34a-b are returned to the “open” position, and the thawing device is ready to receive the next bag of frozen biological material for another thawing operation.

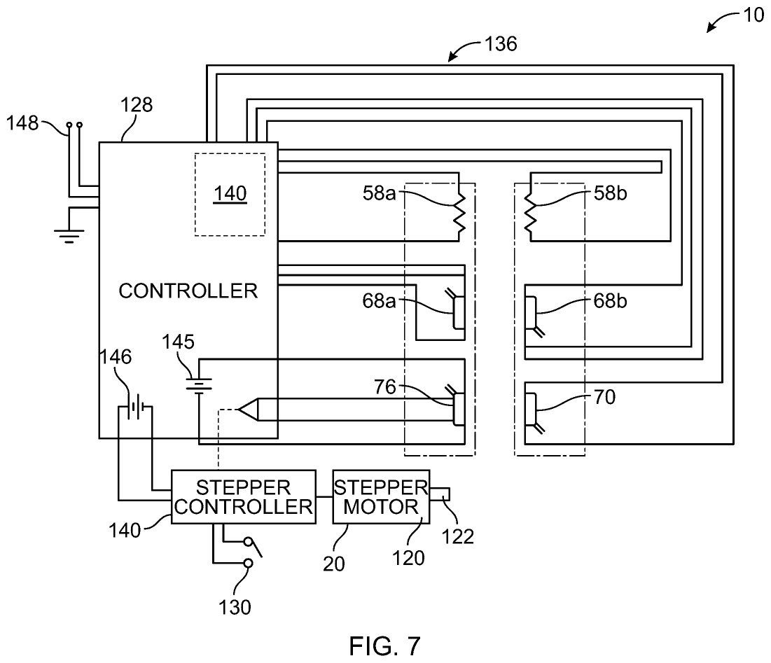

Referring now to FIG. 7, a schematic representation of an exemplary control system 136 implemented in the thawing device 10 according to one embodiment is shown. In the embodiment shown, the thawing device 10 includes multiple, electrically interconnected controllers, such as a programmable controller 138, a heater controller 140 and a stepper motor controller 142, for example, which are configured to control the entire operation of the thawing device 10.

Alternatively, instead of multiple controllers 138, 140, 142 as shown in FIG. 7, the thawing device 10 may be configured with a single multipurpose controller (not shown), as known by those of ordinary skill in the art, to control the entire operation of the thawing device 10.

The main power input 144 of the thawing device 10 may provide a wide range of input voltages, such as input voltages between 100V and 230V, for example or the main power input 144 may provide only a single input voltage, such as 115V or 230V input voltage, for example.

In the embodiment shown, the heater controller 140 may comprise a triac board (not shown) that is electrically connected to the front and rear heaters 58a-b for controlling heating operation of the front and rear heaters 58a-b as described in detail above. Alternatively control of the heating operation of the front and rear heaters 58a-b could be implemented in the single multipurpose controller (not shown). As described above, each of the front and rear heaters 58a-b may comprise a 300W heater which is wired, in one embodiment, with dual circuits for 115V and 230V use.

In the exemplary embodiment, the front and rear heater plate temperature sensors 68a-b are electrically coupled to the controller 138 and comprise resistance temperature detectors (“RTD's”) that are mounted to respective outer faces 60a-b of the front and rear thermally conductive heater plates 44a-b. As described in detail above, the front and rear heater plate sensors 68a-b are provided to monitor the respective temperatures of the front and rear thermally conductive heater plates 44a-b during the thawing operation and control operation of the thawing device as described in detail above.

Still referring to the exemplary embodiment of FIG. 7, the sample temperature sensor 70 is electrically connected to the controller 138 and comprises a thermistor that is supported by the rear thermally conductive heater plate 44b and is configured to sense the temperature of the cryobag 22 during the thawing operation to control operation of the thawing device 10 while the cryobag 22 is being thawed as described in detail above.

As shown in the exemplary embodiment of FIG. 7, the pressure sensor 76 is electrically connected to the controller 138 for controlling operation of the thawing device 10 during a thawing operation as described in detail above. The pressure sensor 76 includes a 5 VDC input 145 which is applied to the pressure sensor 76. In one embodiment as described above, the pressure sensor 76 may comprise a piezo resistive force sensor that is supported by the front thermally conductive heater plate 44a. The pressure sensor 76 is configured to monitor a pressure applied to the inner face 64a of the front thermally conductive heater plate 44a, either by engagement of the front thermally conductive foam pad 62a with one side 40a of the cryobag 22 as shown in FIG. 5, or by engagement of the front thermally conductive foam pad 62a with the rear thermally conductive foam pad 62b (the engagement not shown in the figures).

Still referring to FIG. 7, the stepper motor 120 is electrically connected to the stepper controller 142 which controls operation of the stepper motor 120 in response to the various signals applied to the controller 138 as described in detail above, including the electrical signals generated by the limit switch 130, the pressure sensor 76 and the “OPEN” and “CLOSE” commands, for example. The stepper controller 142 includes a 24V input 146 which is applied to the stepper motor 120.

The limit switch 130 is electrically connected to the stepper controller 142 and operates in two different states as described in detail above, including an “open” state which terminates operation of the stepper motor 120 and a “closed” state which permits operation of the stepper motor 120 as required during a thawing operation.

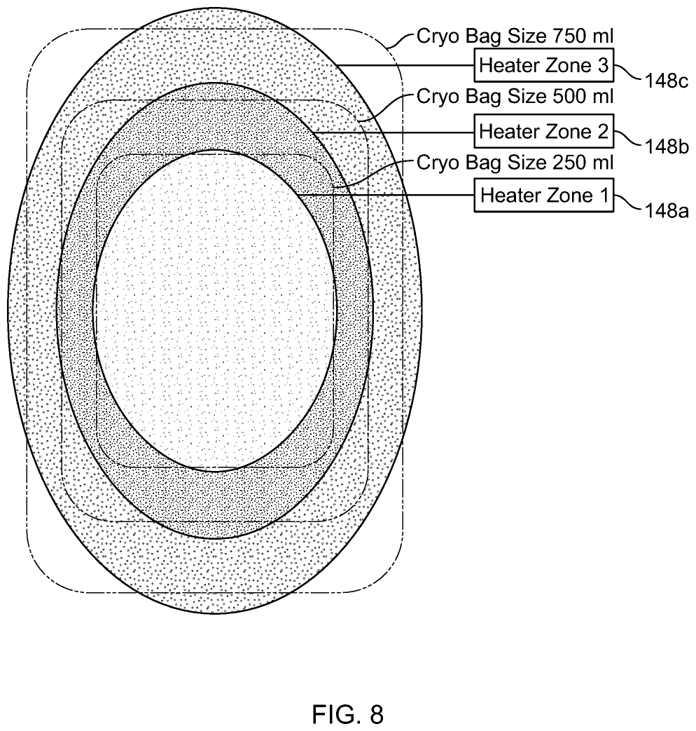

Referring now to FIG. 8, while the exemplary thawing device 10 is shown and described as defining only a single heating zone which is defined by the front and rear heaters 58a-b that are thermally coupled to the respective front and rear thermally conductive heater plates 44a-b, it is contemplated that the front and rear thermally conductive heater plates 44a-b may be provided with multiple banks of respective front and rear heaters (not shown) so as to define one, two or three heating zones 148a-c as shown in FIG. 8.

For example, a first pair of front and rear heaters (not shown) may define a “HEATER ZONE 1” 148a for thawing a smaller size cryobag 22, such as a 250 ml cryobag, for example. A second pair of front and rear heaters (not shown), in combination with the first pair of front and rear heaters (not shown), may define a “HEATER ZONE 2” 148b for thawing a larger size cryobag 22, such as a 500 ml cryobag, for example. Additionally, a third pair of front and rear heaters (not shown), in combination with the first and second pairs of front and rear heaters (not shown), may define a “HEATER ZONE 3” 148c for thawing an even larger size cryobag 22 such as a 750 ml cryobag. While three “HEATER ZONES” 148a-c are shown in FIG. 8, it will be appreciated that more or less “HEATER ZONES” are possible as well.

Further referring to FIG. 8, the thawing device 10 may be provided with multiple sample temperature sensors (not shown), with each of the sample temperature sensors (not shown) being associated with one of the three “HEATER ZONES” 148a-c. Each of the additional sets of front and rear heaters (not shown) would be operated only if a sample temperature sensor (not shown) associated with a particular “HEATER ZONE” detected the presence of a cryo sample in that particular “HEATER ZONE” at the start of a thawing operation. With this particular configuration of multiple “HEATER ZONES” 148a-c, more efficient thawing of the cryobag 22 during a thawing cycle is provided since only the necessary pair or pairs of front and rear heaters (not shown) will be operating depending on the size of the particular cryobag 22.

### ASPECTS

The following Aspects are illustrative only and do not limit the scope of the present disclosure or the appended claims. Any part or parts of any one or more Aspects can be combined with any part or parts of any one or more other Aspects.

Aspect 1. A thawing device configured to thaw frozen liquid contained in at least one bag, comprising: a housing; a first heater plate assembly supported by the housing; at least one first heater thermally coupled to the first heater plate assembly; a second heater plate assembly supported by the housing and opposing the first heater plate assembly to define a bag receiving region between opposing faces of the first heater plate assembly and the second heater plate assembly; at least one second heater thermally coupled to the second heater plate assembly; the thawing device being arranged such that at least one of the first heater plate assembly and the second heater plate assembly is moveable relative to the other of the first heater plate assembly and the second heater plate assembly such that the first heater plate assembly and the second heater plate assembly engage with, and apply heat to, respective opposite sides of the at least one bag containing frozen liquid, with the frozen liquid being thawed to form thawed liquid within the at least one bag when the first and second heaters are operated.

Aspect 2. The thawing device of Aspect 1, wherein the first heater plate assembly comprises a first heater carrier plate and a first thermally conductive heater plate resiliently biased relative to the first heater carrier plate, and further wherein the second heater plate assembly comprises a second heater carrier plate and a second thermally conductive heater plate resiliently biased relative to the second heater carrier plate.

Aspect 3. The thawing device of Aspect 2, wherein the first thermally conductive heater plate is resiliently biased away from the first heater carrier plate, and further wherein the second thermally conductive heater plate is resiliently biased away from the second heater carrier plate.

Aspect 4. The thawing device of Aspect 3, further comprising: a plurality of first resilient members positioned between the first heater carrier plate and the first thermally conductive heater plate; and a plurality of second resilient members positioned between the second heater carrier plate and the second thermally conductive heater plate.

Aspect 5. The thawing device of Aspect 4, wherein the plurality of first resilient members comprises a plurality of first compression springs, and further wherein the plurality of second resilient members comprises a plurality of second compression springs.

Aspect 6. The thawing device of any one of Aspects 1-5, further comprising at least one scissors mechanism operatively coupled to the first heater plate assembly and the second heater plate assembly.

Aspect 7. The thawing device of Aspect 6, further comprising an actuator operatively coupled to the at least one scissors mechanism, wherein the actuator, in a first mode of operation, is operable to move the first heater plate assembly and the second heater plate assembly toward each other and further wherein the actuator, in a second mode of operation, is operable to move the first heater plate assembly and the second heater plate assembly away from each other to disengage from opposite sides of the at least one bag containing the thawed liquid.

It should be understood, however, that an actuator does not have to be coupled to a scissors mechanism. An actuator can be configured to effect motion of any one or more of the first and second heater assemblies. In some embodiments, a first actuator can be associated with the first heater assembly and a second actuator can be associated with the second heater assembly. A thawing device according to the present disclosure can include a single actuator that is associated with any one or more of the first heater assembly and the second heater assembly.

Aspect 8. The thawing device of Aspect 7, further comprising: a pair of scissors mechanisms each operatively coupled to the first heater plate assembly and the second heater plate assembly.

Aspect 9. The thawing device of Aspect 8, wherein the actuator is operatively coupled to each of the pair of scissor mechanisms.

Aspect 10. The thawing device of Aspect 2, wherein the at least one first heater is thermally coupled to the first thermally conductive heater plate, and further wherein the at least one second heater is thermally coupled to the second thermally conductive heater plate.

Aspect 11. The thawing device of any one of Aspects 2-10, further comprising: a first heater foam pad thermally coupled to the first thermally conductive heater plate; and a second heater foam pad thermally coupled to the second thermally conductive heater plate.

Aspect 12. The thawing device of Aspect 2, further comprising: at least one first temperature sensor thermally coupled to the first thermally conductive heater plate; and at least one second temperature sensor thermally coupled to the second thermally conductive heater plate.

Aspect 13. The thawing device of any one of Aspects 2-12, further comprising: at least one sample temperature sensor supported by one of the first and second thermally conductive heater plates.

Aspect 14. The thawing device of any one of Aspects 2-13, further comprising: a pressure sensor supported by one of the first or second thermally conductive heater plates.

Aspect 15. The thawing device of Aspect 7, wherein the actuator comprises a stepper motor; a rotatable screw driven by the stepper motor; and a nut threadably driven by the rotatable screw.

Aspect 16. The thawing device of Aspect 7, wherein the actuator is mounted to pivot about a horizontal axis.

Aspect 17. The thawing device of Aspect 2, further comprising: a plurality of first heaters thermally coupled to the first thermally conductive heater plate to define a plurality of different first heater zones associated with the first thermally conductive heater plate; and a plurality of second heaters thermally coupled to the second thermally conductive heater plate to define a plurality of different second heater zones associated with the second thermally conductive heater plate.

Aspect 18. The thawing device of Aspect 17, further comprising: a plurality of first sample temperature sensors supported by the first thermally conductive heater plate, with each of the plurality of first sample temperature sensors being associated with a respective one of the plurality of different first heater zones; and a plurality of second sample temperature sensors supported by the second thermally conductive heater plate, with each of the plurality of second sample temperature sensors being associated with a respective one of the plurality of different second heater zones.

Aspect 19. A thawing device configured to thaw frozen liquid contained in at least one bag, comprising: a housing; a first heater plate assembly supported by the housing, the first heater plate assembly comprising: a first heater carrier plate and a first thermally conductive heater plate resiliently biased relative to the first heater carrier plate; at least one first heater thermally coupled to the first thermally conductive heater plate; at least one first temperature sensor thermally coupled to the first thermally conductive heater plate; a second heater plate assembly supported by the housing and opposing the first heater plate assembly to define a bag receiving region between opposing faces of the first heater plate assembly and the second heater plate assembly, the second heater plate assembly comprising: a second heater carrier plate and a second thermally conductive heater plate resiliently biased relative to the second heater carrier plate; at least one second heater thermally coupled to the second thermally conductive heater plate; at least one first temperature sensor thermally coupled to the first thermally conductive heater plate; the thawing device being arranged such that at least one of the first heater plate assembly and the second heater plate assembly is moveable relative to the other of the first heater plate assembly and the second heater plate assembly such that the first heater plate assembly and the second heater plate assembly move away from each other to disengage from opposite sides of the at least one bag containing the thawed liquid.

Aspect 20. The thawing device of Aspect 19, further comprising: a first heater foam pad thermally coupled to the first thermally conductive heater plate; and a second heater foam pad thermally coupled to the second thermally conductive heater plate.

Aspect 21. The thawing device of Aspect 19, further comprising: at least one sample temperature sensor supported by one of the first and second thermally conductive heater plates.

Aspect 22. The thawing device of Aspect 19, further comprising: a pressure sensor supported by one of the first or second thermally conductive heater plates.

Aspect 23. The thawing device of Aspect 19, further comprising at least one scissors mechanism operatively coupled to the first heater plate assembly and the second heater plate assembly.

Aspect 24. The thawing device of Aspect 23, further comprising an actuator operatively coupled to the at least one scissors mechanism, wherein the actuator, in a first mode of operation, is operable to move the first heater plate assembly and the second heater plate assembly toward each other and further wherein the actuator, in a second mode of operation, is operable to move the first heater plate assembly and the second heater plate assembly away from each other to disengage from opposite sides of the at least one bag containing the thawed liquid

Aspect 25. The thawing device of Aspect 24, wherein the actuator comprises a stepper motor; a rotatable screw driven by the motor; and a nut threadably driven by the rotatable screw.

Aspect 26. The thawing device of Aspect 24, wherein the actuator is mounted to pivot about a horizontal axis.

Aspect 27. The thawing device of any one of Aspects 19-26, further comprising: a pair of scissors mechanisms each operatively coupled to the first heater plate assembly and the second heater plate assembly.

Aspect 28. The thawing device of any one of Aspects 19-27, further comprising: a plurality of first heaters thermally coupled to the first thermally conductive heater plate to define a plurality of different first heater zones associated with the first thermally conductive heater plate; and a plurality of second heaters thermally coupled to the second thermally conductive heater plate to define a plurality of different second heater zones associated with the second thermally conductive heater plate.

Aspect 29. The thawing device of any one of Aspects 19-28, further comprising: a plurality of first sample temperature sensors supported by the first thermally conductive heater plate, with each of the plurality of first sample temperature sensors being associated with a respective one of the plurality of different first heater zones; and a plurality of second sample temperature sensors supported by the second thermally conductive heater plate, with each of the plurality of second sample temperature sensors being associated with a respective one of the plurality of different second heater zones.

Aspect 30. The thawing device of any one of Aspects 1-18, wherein each of the first and second heater plate assemblies is vertically oriented.

Aspect 31. The thawing device of any one of Aspects 19-29, wherein each of the first and second heater plate assemblies is vertically oriented.

While the invention has been illustrated by the description of various embodiments thereof, and while the embodiments have been described in considerable detail, it is not intended to restrict or in any way limit the scope of the appended claims to such detail. Thus, the various features discussed herein may be used alone or in any combination. Additional advantages and modifications will readily appear to those skilled in the art. The invention in its broader aspects is therefore not limited to the specific details and illustrative examples shown and described. Accordingly, departures may be made from such details without departing from the scope of the general inventive concept.

What is claimed:

1. A thawing device configured to thaw frozen liquid contained in at least one bag, comprising:
a housing;
a first heater plate assembly supported by the housing;
at least one first heater thermally coupled to the first heater plate assembly;
a second heater plate assembly supported by the housing and opposing the first heater plate assembly to define a bag receiving region between opposing faces of the first heater plate assembly and the second heater plate assembly;
at least one second heater thermally coupled to the second heater plate assembly;
the thawing device being arranged such that at least one of the first heater plate assembly and the second heater plate assembly is moveable relative to the other of the first heater plate assembly and the second heater plate assembly such that the first heater plate assembly and the second heater plate assembly engage with, and apply heat to, respective opposite sides of the at least one bag containing frozen liquid, with the frozen liquid being thawed to form thawed liquid within the at least one bag when the first and second heaters are operated.

2. The thawing device of claim 1,
wherein the first heater plate assembly comprises a first heater carrier plate and a first thermally conductive heater plate resiliently biased relative to the first heater carrier plate, and
and further wherein the second heater plate assembly comprises a second heater carrier plate and a second thermally conductive heater plate resiliently biased relative to the second heater carrier plate.

3. The thawing device of claim 2,
wherein the first thermally conductive heater plate is resiliently biased away from the first heater carrier plate, and
further wherein the second thermally conductive heater plate is resiliently biased away from the second heater carrier plate.

4. The thawing device of claim 3, further comprising:
a plurality of first resilient members positioned between the first heater carrier plate and the first thermally conductive heater plate; and
a plurality of second resilient members positioned between the second heater carrier plate and the second thermally conductive heater plate.

5. The thawing device of claim 4,
wherein the plurality of first resilient members comprises a plurality of first compression springs, and
further wherein the plurality of second resilient members comprises a plurality of second compression springs.

6. The thawing device of claim 1, further comprising at least one scissors mechanism operatively coupled to the first heater plate assembly and the second heater plate assembly.

7. The thawing device of claim 6, further comprising an actuator operatively coupled to the at least one scissors mechanism, wherein the actuator, in a first mode of operation, is operable to move the first heater plate assembly and the second heater plate assembly toward each other and further wherein the actuator, in a second mode of operation, is operable to move the first heater plate assembly and the second heater plate assembly away from each other to disengage from opposite sides of the at least one bag containing the thawed liquid.

8. The thawing device of claim 7, further comprising:
a pair of scissors mechanisms each operatively coupled to the first heater plate assembly and the second heater plate assembly.

9. The thawing device of claim 8,
wherein the actuator is operatively coupled to each of the pair of scissor mechanisms.

10. The thawing device of claim 2,
wherein the at least one first heater is thermally coupled to the first thermally conductive heater plate, and
further wherein the at least one second heater is thermally coupled to the second thermally conductive heater plate.

11. The thawing device of claim 10, further comprising:
a first heater foam pad thermally coupled to the first thermally conductive heater plate; and
a second heater foam pad thermally coupled to the second thermally conductive heater plate.

12. The thawing device of claim 2, further comprising:
at least one first temperature sensor thermally coupled to the first thermally conductive heater plate; and
at least one second temperature sensor thermally coupled to the second thermally conductive heater plate.

13. The thawing device of claim 12, further comprising:
at least one sample temperature sensor supported by one of the first and second thermally conductive heater plates.

14. The thawing device of claim 13, further comprising:
a pressure sensor supported by one of the first or second thermally conductive heater plates.

15. The thawing device of claim 7,
wherein the actuator comprises a stepper motor;
a rotatable screw driven by the stepper motor; and
a nut threadably driven by the rotatable screw.

16. The thawing device of claim 7,
wherein the actuator is mounted to pivot about a horizontal axis.

17. The thawing device of claim 2, further comprising:
a plurality of first heaters thermally coupled to the first thermally conductive heater plate to define a plurality of different first heater zones associated with the first thermally conductive heater plate; and
a plurality of second heaters thermally coupled to the second thermally conductive heater plate to define a plurality of different second heater zones associated with the second thermally conductive heater plate.

18. The thawing device of claim 17, further comprising:
a plurality of first sample temperature sensors supported by the first thermally conductive heater plate, with each of the plurality of first sample temperature sensors being associated with a respective one of the plurality of different first heater zones; and
a plurality of second sample temperature sensors supported by the second thermally conductive heater plate, with each of the plurality of second sample temperature sensors being associated with a respective one of the plurality of different second heater zones.

19. A thawing device configured to thaw frozen liquid contained in at least one bag, comprising:
a housing;
a first heater plate assembly supported by the housing, the first heater plate assembly comprising:
a first heater carrier plate and a first thermally conductive heater plate resiliently biased relative to the first heater carrier plate;
at least one first heater thermally coupled to the first thermally conductive heater plate;
at least one first temperature sensor thermally coupled to the first thermally conductive heater plate;
a second heater plate assembly supported by the housing and opposing the first heater plate assembly to define a bag receiving region between opposing faces of the first heater plate assembly and the second heater plate assembly, the second heater plate assembly comprising:
a second heater carrier plate and a second thermally conductive heater plate resiliently biased relative to the second heater carrier plate;
at least one second heater thermally coupled to the second thermally conductive heater plate;
at least one first temperature sensor thermally coupled to the first thermally conductive heater plate;
the thawing device being arranged such that at least one of the first heater plate assembly and the second heater plate assembly is moveable relative to the other of the first heater plate assembly and the second heater plate assembly such that the first heater plate assembly and the second heater plate assembly move away from each other to disengage from opposite sides of the at least one bag containing the thawed liquid.

20. The thawing device of claim 19, further comprising:
a first heater foam pad thermally coupled to the first thermally conductive heater plate; and
a second heater foam pad thermally coupled to the second thermally conductive heater plate.

21. The thawing device of claim 19, further comprising:
at least one sample temperature sensor supported by one of the first and second thermally conductive heater plates.

22. The thawing device of claim 19, further comprising:
a pressure sensor supported by one of the first or second thermally conductive heater plates.

23. The thawing device of claim 19, further comprising at least one scissors mechanism operatively coupled to the first heater plate assembly and the second heater plate assembly.

24. The thawing device of claim 23, further comprising an actuator operatively coupled to the at least one scissors mechanism, wherein the actuator, in a first mode of operation, is operable to move the first heater plate assembly and the second heater plate assembly toward each other and further wherein the actuator, in a second mode of operation, is operable to move the first heater plate assembly and the second heater plate assembly away from each other to disengage from opposite sides of the at least one bag containing the thawed liquid.

25. The thawing device of claim 24,
wherein the actuator comprises a stepper motor;
a rotatable screw driven by the motor; and
a nut threadably driven by the rotatable screw.

26. The thawing device of claim 24,
wherein the actuator is mounted to pivot about a horizontal axis.

27. The thawing device of claim 19, further comprising:
a pair of scissors mechanisms each operatively coupled to the first heater plate assembly and the second heater plate assembly.

28. The thawing device of claim 19, further comprising:
a plurality of first heaters thermally coupled to the first thermally conductive heater plate to define a plurality of different first heater zones associated with the first thermally conductive heater plate; and
a plurality of second heaters thermally coupled to the second thermally conductive heater plate to define a plurality of different second heater zones associated with the second thermally conductive heater plate.

29. The thawing device of claim 28, further comprising:
a plurality of first sample temperature sensors supported by the first thermally conductive heater plate, with each of the plurality of first sample temperature sensors being associated with a respective one of the plurality of different first heater zones; and
a plurality of second sample temperature sensors supported by the second thermally conductive heater plate, with each of the plurality of second sample temperature sensors being associated with a respective one of the plurality of different second heater zones.

30. The thawing device of claim 1,
wherein each of the first and second heater plate assemblies is vertically oriented.

31. The thawing device of claim 19,
wherein each of the first and second heater plate assemblies is vertically oriented.
